(19) United States
(12) Patent Application Publication
(10) Pub. No.: US 2017/0165438 A1
(43) Pub. Date: Jun. 15, 2017

(54) Single Use Atomizer Designed To Minimize Chances of Contamination

(71) Applicant: Microbiome Solutions, LLC, Byron Center, MI (US)

(72) Inventor: Simon Wright, Urbandale, IA (US)

(21) Appl. No.: 15/379,787

(22) Filed: Dec. 15, 2016

Related U.S. Application Data
(60) Provisional application No. 62/267,697, filed on Dec. 15, 2015.

(51) Int. Cl. A61M 11/06; A61M 11/02; B05B 7/24; A61M 3/02

(52) U.S. Cl. CPC A61M 11/06; A61M 3/02; A61M 11/02; B05B 7/2408; B05B 12/002

(57) ABSTRACT

The single use atomizer retains the look, feel, heft, and operation of the multi-use atomizers preferred by most doctors by replacing the standard multi-dose reservoir with a modified reservoir and shortening the inlet tube. The modified reservoir is equipped to accept a single-dose receptacle and with means to associate the single-dose receptacle with the standard nozzle, applicator, and trigger.

### CROSS REFERENCE TO RELATED APPLICATION

This application claims benefit from U.S. Patent and Trademark Office Provisional Application No. 62/267,697 filed on Dec. 15, 2015.

### BACKGROUND

Ear nose and throat doctors and surgeons have long-used nasal irrigation and/or application to deliver a variety of therapeutic agents. One very popular method comprises atomizing the therapeutic agent.

Although there are several pieces of equipment available to atomize a fluid for therapeutic application, a favorite among many is the DeVilbiss atomizer such as model 163. The DeVilbiss model, like many others, comprises a reservoir, a nozzle body, means to atomize fluid in air, an applicator, and a trigger and a source of pressurized air. Said means to atomize air comprises an arrangement that allows pressurized air to pass over an inlet tube in the reservoir, creating a venturi effect which draws a fluid from the reservoir up the inlet tube and into means to atomize the fluid, through the nozzle and applicator. The trigger is associated with the source of pressurized air; upon activation of the trigger typically opening a valve that allows pressurized air to escape its container, the pressurized air is guided by conduit to move into said means to atomize. As the air passes through the atomizer over the inlet tube, it creates a venturi effect drawing the fluid from the receptacle up to the atomizer where it is atomized and forced through the nozzle body and, finally, the applicator. The size of the conduits and the pressure of the air dictate how much fluid is atomized and delivered over a unit of time.

In prior art, the reservoir has contained multiple doses of the therapeutic agent to be delivered. The nozzle and applicator are routinely cleaned and disinfected as they are the only parts in contact with the patient. Recent studies (see “Multi-use venturi nasal atomizer contamination in a clinical rhinologic practice”, American Journal of Rhinology 18(3):151-6, May 2004 and “The risk of patient cross-contamination from venture-principle atomizers”, ORL Head Neck Nurs. 2005 Spring 23(2):25-7 and “Venturi atomizers as potential sources of patient cross-infection”, Am J Infect Control. 2003 November; 31(7):441-4) have suggested, however, that the reservoir may become contaminated, raising the possibility of cross-contamination from patient to patient.

The widely accepted use of the DeVilbiss model and several other similar models may be attributed to a number of factors; for one, it is often the atomizer used by medical schools and hospitals. Its reservoir is made of glass; the trigger and nozzle apparatus are constructed of steel, both materials fairly resistant to absorbing or deposition of contaminants. Pressurized air is necessary to achieve the level of atomization required for application and for uniform delivery in turn dictating a certain level of strength required in the construction of atomizers. Further, the DeVilbiss model and others like it, have a certain weight, touch, feel, and trigger tension many doctors have used for years and would prefer not to give up.

What was needed was an atomizer having, generally, the same touch and feel of the current air-pressurized atomizers but designed in such a way as to eliminate reservoir contamination.

### SUMMARY OF THE INVENTION

The present invention comprises a modified reservoir for use with a standard atomizer and, preferably, a single-dose receptacle. While the idea of single dose receptacles are not new, this invention is specifically designed to retain and maintain the look, feel, weight and operational features of the DeVilbiss atomizer models and those similar thereto which are preferred by practitioners. Embodiments comprise replacing the standard reservoir for an atomizer with a reservoir that is of the same material, overall shape and weight which has been modified with particularity to receive a single dose receptacle. The single dose receptacle is inserted, at least partially, in the modified reservoir in a manner to provide fluid connection with the inlet tube that transports fluid to the atomizer. After use of the fluid in the single dose receptacle, the receptacle is removed and discarded, another is inserted. By modifying the standard reservoir to accept the single dose receptacle, the overall operation, weight, and feel of the atomizer is retained, maintenance activity is reduced and the possibility of contamination of the reservoir is removed.

To accomplish the objectives of the invention, the modified reservoir includes an alcove on its outer cylindrically shaped surface or a slot into which the single-dose receptacle is at least partially inserted. The alcove or slot is sized so as to allow the single-dose receptacle to be pushed upward, or, perhaps, inward to fluidly connect with the inlet tube. The inlet tube is, in turn, fluidly connected to the atomizer. Connecting means are provided to physically secure the single-dose receptacle in the alcove and to the inlet tube in fluid communication with the atomizer. The alcove is sized to allow the single-dose receptacle to be easily inserted and connected, as well as easily removed. The same principal holds for sizing of a slot as an alternative. In general, the size of the alcove or slot must be such that the user is able to easily insert the single-dose receptacle and move it upward or inward, depending on the internal arrangement, to allow connection with the inlet tube. This may require vertical and/or horizontal clearance in the slot or alcove adequate to allow an adult finger or fingers to be used to insert and then connect the single-dose receptacle. The connection with the inlet tube may be a friction fit, a threaded relationship, or valved relationship, or may employ other known means to provide a sealed, fluid connection.

### BRIEF DESCRIPTION OF THE DRAWINGS

FIG. 1 is a cut-away view of a prior art standard atomizer.

FIG. 2 is a cut-away view of an atomizer of the present invention loaded with a single dose receptacle.

FIG. 3 is a side view of a single dose receptacle.

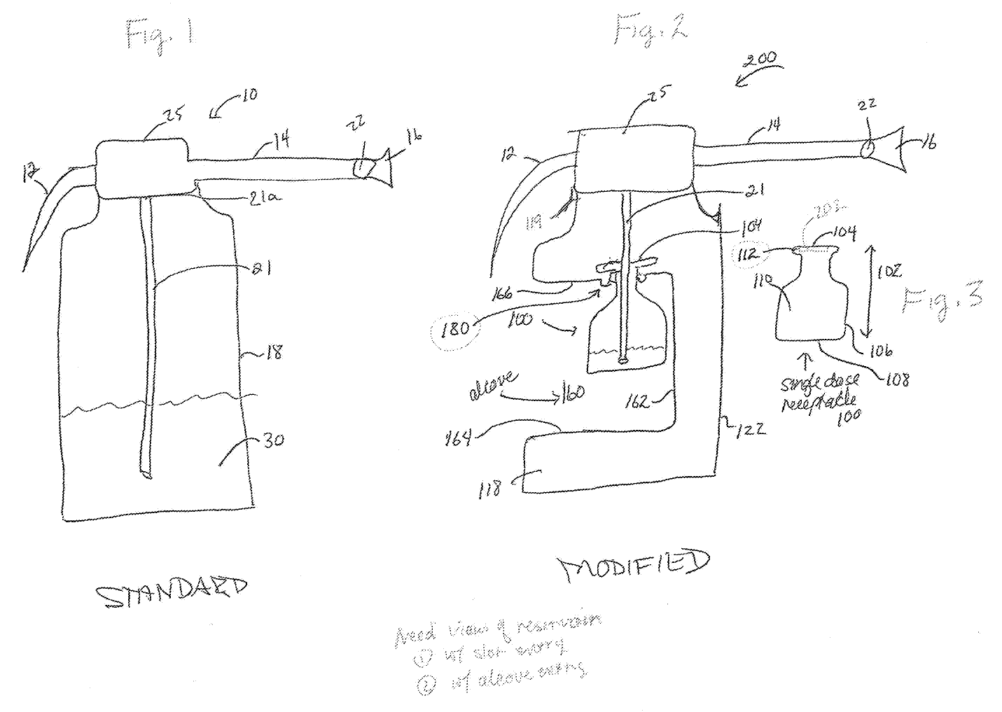

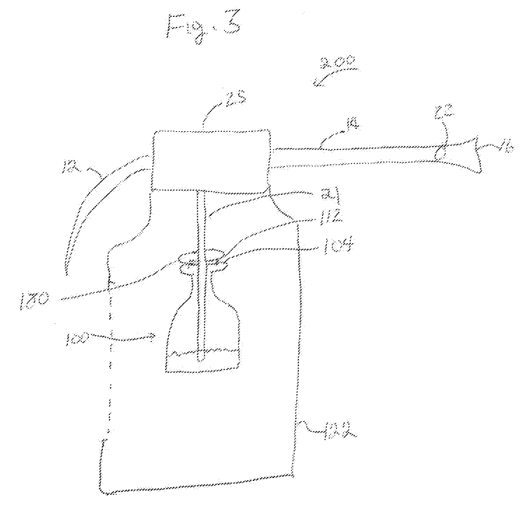

### DETAILED DESCRIPTION OF THE PRESENT INVENTION

A standard atomizer 10 comprises a trigger 12 associated with an outlet 8 for a pressurized gas 100 and an atomizing apparatus 20. The atomizing apparatus 20 may comprise an atomizing nozzle 22 fluidly connected to an atomizer conduit 14 and may or may not be equipped with means to close the connection. The atomizer 10 further comprises an applicator 16 connected to the atomizer conduit 14, and a standard reservoir 18 capable of containing a volume of fluid 30 for several doses. The standard reservoir is associated with an atomizer body 25 in a generally airtight manner for a purpose to be described. Often that association is accomplished through mating threads on the applicator and the standard reservoir. In other embodiments, the applicator and the standard reservoir are associated by a pressure or snap fit which is facilitated by elastic and flexible ridges, tabs or rings.

According to prior art, an inlet tube 21 connects with the atomizing apparatus 20 and extends through the atomizer body 25 into the standard reservoir 18. The atomizing apparatus 20 comprises an opening 21a of the inlet tube 21 and the atomizer body 25 where, upon activation of the trigger 12, the pressurized gas 90 flows past the opening of the inlet tube 21a and draws fluid 30 into it from the reservoir via the generally airtight seal between standard reservoir 18 and the atomizer body 25. This arrangement facilitates suction for drawing up the fluid 30 in the inlet tube 21 through its opening 21a, through the nozzle conduit 14 to the atomizer 10 where it is mixed with the pressurized gas 90 by the venturi effect and thereafter delivered by the applicator 16. Activation by the trigger 12, therefore, delivers the fluid 30 through the atomizer body 25 to the atomizing apparatus 20 where it is atomized with the pressurized gas 90 and delivered in atomized form through the applicator 16.

In the present invention, the modified reservoir 118 has been constructed to function more like a cartridge holder for use with disposable, single-use receptacles 100 preloaded with the fluid 30 in a volume constituting a single dose. An alternate embodiment may comprise disposable receptacles having several doses. Replacing the standard reservoir 18 with a modified reservoir 118 requires no or only minimal alteration of the standard atomizer 10 and mechanics described above.

A single-dose receptacle 100 comprises a height 102, a top 104, and a volume 110 enclosed by a wall 106. The volume 110 may be adequate for a single dose or a bit larger to hold a few doses.

In an embodiment, the modified reservoir 118 comprises a connection end 119, which is associated with the atomizer body 25 in a generally airtight manner for a purpose to be described. The modified reservoir 118 further comprises a bottom 120, and a generally cylindrical wall 122 as is common in the standard reservoir 18. Cylindrically walled vessels are easier to clean because there are fewer corners in which material can become lodged. However, like the standard reservoir, the shape of the sidewall of the modified reservoir may optionally be of rectangular or angular volume. In a first embodiment, one portion of the wall 122 of the modified reservoir 118 comprises an alcove 160. The alcove 160 includes an inner surface 162 and, preferably, a bottom surface 164. The inner surface 162 of the alcove 160 may be a single curved surface or may comprise a plurality of sides. An inner top surface 166 of the alcove 160 comprises means to associate 180 the single-dose receptacle 100 in a generally airtight manner and further comprises an opening 182 through which the inlet tube 21 extends. The top 104 of the single dose receptacle preferably includes means 112 complementary to means to associate 180. In one embodiment, the means to associate 180 and complementary means 112 together comprise a generally airtight friction fit. In another, the fit is a threaded fit. In a second embodiment, the wall 122 of the modified reservoir 118 comprises a slot or opening 200 into which the single-dose receptacle 100 may be inserted and then associated with the means to associate 180 the single-dose receptacle 100 in a generally airtight manner as previously described. Said means to associate 180 may be associated with the inlet tube 21. This alternative embodiment may or may not include bottom surface 164 or inner surface 162 as was described relative to alcove 160.

In an embodiment, the alcove 160 comprises a height 162 sized to closely fit the height 102 of the single-dose receptacle 100. In this embodiment, the inner top surface 166 comprises guides 200 and 202 between which the top 104 is placed and then slid into the alcove 60. In a second embodiment, the alcove 60 comprises a height greater than the height 102 of the single-dose receptacle 100; the single-dose receptacle 100 is inserted into the alcove 60 and then moved upward to engage the means to associate 180 by the complementary means 112. In the third embodiment comprising the slot 200, its height may be any dimension that allows the receptacle 100 to be inserted. In each of the embodiments the connection end 119 of the modified reservoir 118 is associated with the atomizer body 25 by the inlet tube 21 extending downward into the single-dose receptacle 100. Alternatively the atomizer body 25 is associated with the modified reservoir 118; a single-dose receptacle 100 may further comprise a pierce-able cover 202 over the top 104 such that when the receptacle 100 is inserted into the alcove 60 and then moved upward to engage the means to associate 180 by the complementary means 112, the inlet tube 21 (which comprise a sharpened end 23 or may include a moveable piercing component) pierces the cover 202 to extend into the volume 110. Upon completion of use, the single receptacle 100 is removed by reversing the association routine and discarded or, if reusable, is sanitized and re-loaded.

1. An atomizer comprising an atomizer body, a modified reservoir having generally the same outer shape and size of the DeVilbiss model 163 said modified reservoir comprising means to accommodate substantially all of a single dose receptacle in said modified reservoir, a single dose receptacle and means to fluidly associate said single dose receptacle with said atomizer body, said single dose receptacle comprising a top and a bottom.

2. The atomizer of claim 1 wherein the modified reservoir comprises a connection end for association with the atomizer body.

3. The atomizer of claim 1 further comprising an inlet tube, said means to accommodate comprising an opening in the outer shape.

4. The atomizer of claim 3 further comprising a trigger, a nozzle conduit, and an applicator

5. The atomizer of claim 3 wherein said single dose receptacle further comprises a pierce-able cover.

6. The atomizer of claim 4 wherein said means to fluidly associate said single dose receptacle with the atomizer body comprises means to associate on the modified reservoir and complementary means to associate on the single dose receptacle.

7. The atomizer of claim 6 wherein the said means to associate on the modified reservoir comprise a track and said complementary means on the single dose receptacle comprises a lip sized to fit within and engage said track.

8. The atomizer of claim 1 wherein the means to fluidly associate said single dose receptacle and the modified reservoir body comprise a friction fit.

9. The atomizer of claim 1 wherein the means to fluidly associate said single dose receptacle and the modified reservoir body comprise threads on one of the single dose receptacle and the modified reservoir body and grooves on the other whereby the threads are sized to fit and turn into the groove, thereby removably associating the single dose receptacle and the modified reservoir body.

10. The atomizer of claim 1 said modified reservoir body of claim 1 comprising an alcove having an inner surface, a bottom surface, and an inner top surface said inner top surface.

11. The atomizer of claim 10, further comprising an inlet tube.

12. An atomizer assembly comprising an atomizer body, a modified reservoir comprising shape and size similar to a reservoir on a DeVilbiss Model 163, and a single dose receptacle said modified reservoir comprising means to accommodate said single dose receptacle and facilitate fluid communication between the single dose receptacle and the atomizer body, said single dose receptacle comprising a top and a bottom said means to accommodate the receptacle comprising an opening in the modified reservoir sized to allow insertion of at least a portion of the single dose receptacle, and means to fluidly associate said single dose receptacle with said atomizer body.

13. The assembly of claim 12 said means to facilitate fluid communication between the single dose receptacle and the atomizer body comprising an inlet tube extending between the atomizer body and the top of the single dose receptacle.

14. The assembly of claim 13 further comprising means to associate on the modified reservoir and complementary means to associate on the single dose receptacle.

15. A method of using the atomizer assembly of claim 13 wherein said opening comprises a lower portion below the inlet tube and an upper portion, said receptacle inserted in the lower portion then elevated to the upper portion thereby inserting the inlet tube in the single dose receptacle, thereafter associating the means to associate on the modified reservoir with the complementary means to associate on the single dose receptacle.
